(19) United States
(12) Patent Application Publication
(10) Pub. No.: US 2014/0377189 A1
(43) Pub. Date: Dec. 25, 2014

(54) PULMONARY ADMINISTRATION OF ROTIGOTINE

(71) Applicant: MAP Pharmaceuticals, Inc., Irvine, CA (US)

(72) Inventors: Libo WU, Mountain View, CA (US); Wiwik WATANABE, Union City, CA (US)

(21) Appl. No.: 14/307,998

(22) Filed: Jun. 18, 2014

Related U.S. Application Data
(60) Provisional application No. 61/837,000, filed on Jun. 19, 2013.

(51) Int. Cl. A61K 9/00; A61K 31/20; A61K 31/194; A61K 31/381; A61K 31/19

(52) U.S. Cl. CPC A61K 9/008; A61K 31/381; A61K 31/19; A61K 31/194; A61K 31/20
USPC 424/45

(57) ABSTRACT

Provided herein are methods and compositions for producing formulations systemic delivery of dopamine agonists via the oral inhalation route. Specifically, provided herein are methods and compositions for a stable solution formulation of rotigotine that is suitable for administration via oral inhalation. Such methods and compositions are useful in the treatment or amelioration of one or more Parkinson's disease symptom(s).

### CROSS-REFERENCE

This application claims priority under 35 U.S.C. §119(e) from U.S. Provisional Application Ser. No. 61/837,000, filed Jun. 19, 2013, which is hereby incorporated by reference in its entirety.

### TECHNICAL FIELD OF THE INVENTION

The invention relates to new compositions and methods of treating Parkinson's disease. More specifically, the compositions and methods described herein are in the field of orally inhaled aerosol formulations. Specifically, compositions and methods that allow for the orally inhaled administration of rotigotine formulations are described.

### BACKGROUND OF THE INVENTION

Parkinson's disease is characterized by motor symptoms such as tremor, slowed ability to start and continue movements (bradykinesia), muscle rigidity, gait dysfunction and postural instability. All Parkinson's disease patients experience one or more of these symptoms, which progressively worsens with time. Researchers have identified that a degeneration of dopaminergic neurons in the substantia nigra area of the brain and degeneration of dopaminergic fibers as the primary pathophysiological mechanisms in Parkinson's disease. Additionally, researchers believe that other neurotransmitter systems such as serotonergic and glutamatergic systems are also involved in the disease process.

Rotigotine (5,6,7,8-tetrahydro-6-[propyl-[2(-thienyl)ethyl]amino]-1-naphthalenol, and its pharmaceutically acceptable salts have been known to be administered to patients through mostly transdermal delivery systems (see e.g., U.S. Pat. No. 7,413,747 and U.S. Pat. No. 6,884,434) and intranasal administration (see e.g., U.S. Pat. No. 7,683,040). Dopamine D2 agonists, such as rotigotine, may be effective agents in treating the symptoms of Parkinson's disease and other diseases for which an increase of dopamine levels may be beneficial, such as, but not limited to, restless leg syndrome (RLS). However, due complications from low bioavailability and the high systemic exposure of these dopamine D2 agonists, the development of safe and effective pharmaceutical formulations of dopamine D2 agonists, such as rotigotine, are needed.

Aerosols are increasingly being used for delivering medication for therapeutic treatment to the lungs. This type of pulmonary drug delivery depends on the subject inhaling an aerosol through the mouth and throat so that the drug substance can reach the lungs (i.e., oral inhalation). For drugs that are systemically active (e.g., the intended active site is not the lungs), inhalation delivery to the alveolar region of the lung is preferred.

There are several advantages for aerosol delivery of systemically active drugs to the lungs. One major advantage is the fast absorption through the lung epithelium and delivery of the drug into systemic circulation. This advantage is particularly suitable for drugs that require a fast onset of action.

Rotigotine has generally been formulated for transdermal delivery. However, there are consistency issues relating to transdermal delivery of rotigotine. Others have also described an intranasal formulation of rotigotine (U.S. Pat. No. 7,683,040). However, given the common impairment of motor control in Parkinson's disease patients, intranasal administration of rotigotine may be challenging and could require administration by a healthcare professional or in a hospital setting. Additionally, there would be complications due to consistency of dose through intranasal administration (e.g., insufflation) such as loss of the formulation on the nasal septum, where the formulation does not reach the intended nasal mucosa. Also, there may be significant loss of the formulation due to dose dripping down the throat and into the stomach. Oral inhalation delivery of dopamine D2 agonists such as rotigotine would overcome these difficulties and/or disadvantages.

There is a significant need for stable orally inhaled dopamine D2 agonists, such as rotigotine. The present invention satisfies this need.

### SUMMARY OF THE INVENTION

The invention encompasses methods and compositions of a pharmaceutical aerosol formulation comprising a dopamine agonist; a propellant; a cosolvent and where the aerosol formulation is a stable solution formulation. In another aspect, the pharmaceutical aerosol formulation is stable at room temperature for at least a week. In another aspect, dopamine agonist in the pharmaceutical aerosol formulation is rotigotine or a pharmaceutically acceptable salt thereof. In yet another aspect, the rotigotine in the pharmaceutical aerosol formulation is selected from the group consisting of rotigotine glycolate, rotigotine lactate, rotigotine maleate, rotigotine palmitate, rotigotine pamoate, rotigotine propionate, and rotigotine stearate. In another aspect, the rotigotine in the pharmaceutical aerosol formulation is rotigotine maleate. In another aspect the propellant is selected from the group consisting of 1,1,1,2-tetrafluoroethane and 1,1,1,2,3,3,3-heptafluoropropane. In another aspect, the propellant is 1,1,1,2,3,3,3-heptafluoropropane. In another aspect, the propellant is a mixture of 1,1,1,2-tetrafluoroethane and 1,1,1,2,3,3,3-heptafluoropropane. In another aspect the cosolvent is selected from the group consisting of ethanol, propylene glycol, polyethylene glycol, and water. In another aspect, the cosolvent is ethanol. In another aspect, the pharmaceutical aerosol formulation is administered to a patient using a pressurized metered dose inhaler. In another aspect, the pressurized metered dose inhaler is breath-actuated. In another aspect, the pharmaceutical aerosol formulation is administered to a patient using a nebulizer. In another aspect, the concentration of the dopamine agonist in the pharmaceutical aerosol formulation is at least 1 mg/mL.

According to another aspect of the invention, the invention relates to a pharmaceutical aerosol formulation comprising rotigotine or a pharmaceutically acceptable salt thereof, a propellant, and a cosolvent, wherein the aerosol formulation is a stable solution formulation and wherein the propellant is 1,1,1,2,3,3,3-heptafluoropropane and the cosolvent is ethanol. In another aspect, the rotigotine is rotigotine maleate.

According to another aspect of the invention, the invention relates to a pharmaceutical aerosol formulation consisting of rotigotine maleate, a propellant, and a cosolvent, wherein the aerosol formulation is a stable solution formulation.

### DETAILED DESCRIPTION OF THE INVENTION

This detailed description of the invention is divided into sections for the convenience of the reader. Section I provides definitions of terms used herein. Section II provides a description of methods and compositions of orally-inhaled dopamine agonists. Section III provides a description of oral inhalation delivery systems. Section IV discloses examples that illustrate the various aspects and embodiments of the invention.

### I. DEFINITIONS

Unless defined otherwise, all technical and scientific terms used herein have the meaning commonly understood by a person skilled in the art to which this invention belongs. As used herein, the following terms have the meanings ascribed to them unless specified otherwise.

“Active pharmaceutical ingredient” or “API” refers to active chemical(s) used in the manufacturing of drugs. Another term synonymous with API is “bulk drug substance”. It is understood that API refers to the active pharmaceutical ingredient including any and all appropriate salts, hydrates, solvates, polymorphs, prodrugs, ion pairs, and metabolites thereof.

“Drug composition” or “drug formulation” refers to a composition comprising at least one API and at least one additional composition.

“Excipient” refers to pharmaceutically acceptable carriers that are relatively inert substances used to facilitate administration or delivery of an API into a subject or used to facilitate processing of an API into drug formulations that can be used pharmaceutically for delivery to the site of action in a subject. Non-limiting examples of excipients include stabilizing agents, surfactants, surface modifiers, solubility enhancers, buffers, encapsulating agents, antioxidants, preservatives, nonionic wetting or clarifying agents, viscosity increasing agents, and absorption-enhancing agents.

“Hydrofluorocarbon” refers to hydrofluoroalkanes (HFAs). In recent years, HFAs have replaced chlorofluorocarbons (CFCs) as propellants due to environmental issues concerning the impact of CFCs on the earth's ozone layer. Examples of hydrofluoroalkane propellants include of 1,1,1,2-tetrafluoroethane (referred to as HFA134a) and 1,1,1,2,3,3,3-heptafluoropropane (referred to as HFA 227).

“Particulate API” refers to an API that is manufactured at a desired particle size or particles of a desired particle size range.

“QT Prolongation” refers to a prolonged period between the Q wave and the T wave in an electrocardiogram (heart's electrical cycle). In general, the QT interval represents electrical depolarization and repolarization of the right and left ventricles of the heart. QT prolongation can occur as a side-effect of certain medication(s) and is a biomarker for ventricular tachyarrhythmias and is a risk factor for sudden death.

“Solution” refers to a homogeneous mixture composed of only one phase. As applied to the invention, the API is dissolved in a suitable solvent or diluent to form a stable solution.

“Stabilized pharmaceutical formulation” refers to a pharmaceutical formulation that exhibits physical and chemical stability in which the physical and chemical composition characteristics of the formulation do not change significantly due to the effects of time and temperature.

“Surface modifier” refers to organic or non-organic pharmaceutically acceptable excipients that are typically added to a drug formulation to alter formulation performance. Such alterations in performance include reduction, minimization or elimination of aggregation or agglomeration of particle of a drug. Surface modifiers include, but are not limited to, polymers, low molecular weight oligomers, and surfactants.

“Suspension” refers to a chemical system composed of components in a medium where the components are larger than those comprising the medium. Components of a suspension can be evenly distributed, for example by mechanical means, however, the components will settle out of the medium under the influence by gravity.

“Unit dosage form” refers to a physically discrete unit suitable as unitary dosages for an individual, each unit containing a predetermined quantity of active material calculated to produce a desired therapeutic effect, in association with a suitable pharmaceutical carrier, diluent, solvent, or excipient. These unit dosage forms can be stored in suitable packaging in single or multiple unit dosages and may also be further sterilized and sealed.

It is to be understood that this invention is not limited to particularly exemplified drug particles, formulations, or manufacturing processes parameters as such, may vary. It is also to be understood that the technical terminology used herein is for the purpose of describing particular embodiments of the invention only, and is not intended to be limiting.

### II. METHODS AND COMPOSITIONS OF ORALLY-INHALED DOPAMINE AGONISTS

### Dopamine D2 Agonists

Because there are no proven disease-modifying therapies for Parkinson's disease, the primary goal of medical treatment is managing the multitude of motor and behavioral symptoms associated with the disease and improving patient's quality of life. L-dopa or levodopa is currently the “gold standard” for the management of motor symptoms of Parkinson's disease. L-dopa (L-3.4-dihydroxyphenylalanine) is the precursor to the neurotransmitters dopamine, norepinephrine and epinephrine. However, there are many adverse side-effects associated with chronic administration of levodopa.

One of the many symptoms of Parkinson's disease that many patients face is “freezing”. Freezing is the temporary, involuntary inability to move. Freezing may occur at any time and some patients are more prone to freezing than others. In many cases, patients may experience freezing of gait when the patient is due for the next dose of dopamine precursor therapy (e.g., levodopa), such a period is referred to as an “off period”. To alleviate freezing, current treatment calls for the increase of dopaminergic medication(s) in order to avoid the off period. However, because administration of dopaminergic medications are usually by oral therapy (e.g., pill or tablet), the time to wait for the medication to become bioavailable is quite lengthy (usually about 1-2 hours). Additionally, the administration of more dopaminergic medication such as levodopa may increase the side effects such as end of dose deterioration of function, on/off period oscillations, increase in freezing during movement, other motor response complications, drug resistance, dyskinesia, serotonin depletion, and dopamine dysregulation.

The ideal candidate for rescue treatment of Parkinson's disease symptoms should have a fast onset and short half-life; be effective in treating the Parkinson's off period; reduced nausea as to eliminate the need for an antiemetic; non-invasive and convenient route of administration; and minimal drug interactions (e.g., can be co-administered with levodopa).

The current rescue treatment for Parkinson's disease symptoms is apomorphine. Apomorphine is a morphine-derived is usually administered through injection. Administration through injection has the advantage of having a fast onset of action. Also, apomorphine has a short half-life and is effective in treating the off periods. However, its drawbacks are that injection is invasive and not convenient. Additionally apomorphine causes nausea, which requires co-administration with an antiemetic, and apomorphine may cause QT prolongation.

Dopamine agonists have been used for more than two decades as adjuncts to levodopa for patients suffering from levodopa-related motor response complications. Adjunct dopamine agonist therapy enables a lower dose of levodopa, which can ameliorate levodopa-induced side effects. The addition of a dopamine agonist can also help to extend the patient's “on” period and to relieve the effects of an off period. One such dopamine agonist is rotigotine. Currently rotigotine is available as a transdermal patch. However, drawbacks exist for transdermal delivery of rotigotine including dosing issues and patient compliance. The present invention addresses both of these problems.

An orally inhaled rotigotine formulation may be advantageous as a rescue therapy for Parkinson's off periods. Rotigotine's relatively short half-life is ideal for rescue therapy. Rotigotine is a dopamine D2 agonist and is a proven therapy for managing motor symptoms associated with Parkinson's disease. Rotigotine is also highly lipophilic which makes it suitable for rapid penetration through the lung epithelial barrier and the blood brain barrier. Additionally, there seems to be less adverse side-effects associated with rotigotine than other dopamine agonists such as ropinerole and pramipexole.

Systemic delivery via the oral inhalation route (and thereby through absorption in the lungs into systemic circulation) provides several advantages when the primary intended site of action of the drug is the brain. One advantage is the very rapid absorption by the lung and delivery into systemic circulation. Once absorbed by the lungs, the drug will enter into the pulmonary artery and then to the carotid artery to the brain. Once in the brain, the drug can cross the blood-brain barrier and be delivered to the intended site of action. This targeted delivery to the brain avoids first pass metabolism and avoids any enzyme degradation that may occur. Because the brain (via the carotid) is one of the first major organ that is engaged via this route of systemic circulation, oral inhalation also can minimize potential systemic side effects and may lower the dose required for efficacy in a subject.

Another advantage for an orally inhaled rotigotine formulation is the relatively fast onset of action for drugs that are administered to the lungs for systemic delivery to brain (one site of action). Compared to oral administration through a pill or tablet which has an onset of action of between 1-2 hours, oral inhalation/pulmonary administration for systemic delivery to the brain has an onset of action usually of less than 20 minutes after administration. Because of the rapid onset of action achieved through pulmonary administration of systemically active drugs, this method of delivery is preferred for acute treatment of symptoms such as rescue from Parkinson's freezing event(s).

Also, unlike oral administration, pulmonary administration through oral inhalation bypasses the gastrointestinal tract and thus also avoids enzymatic degradation, problems with gastric stasis (in some diseases) and inconsistent absorption rates, giving the patient a more consistent delivery of the drug. Unlike IV or IM injection, pulmonary administration through oral inhalation is convenient, non-invasive, self-administrable and no hospitalization is required.

In some embodiments, the orally inhaled active pharmaceutical ingredient (API) is rotigotine. In other embodiments, the API formulation is a rotigotine maleate salt solution.

### Aerosol Formulations

Aerosol formulations of an API may be in either a suspension or a solution. Particulate active pharmaceutical ingredient (API) that are of an acceptable particle size for delivery to the lungs in a suspension aerosol formulation may be generated in a variety of manner. For illustrative purposes, API particles may be generated from the bulk API by attrition processes such as grinding, micronizing, milling or the like. API particles may also be generated through a multiphase precipitation process such as spray drying, solution precipitation, in situ precipitation, volume exclusion precipitation, supercritical extraction/precipitation, lyophilization, or the like. API particles for use in aerosols are generally manufactured to a size of about 0.05 microns to about 10 microns, of about 0.1 microns to about 5 microns, of about 0.5 microns to about 3 microns, and of about 1 micron to about 3 microns. In various embodiments, the active pharmaceutical ingredient has a particle size in the range of about 0.5 microns to about 3 microns. In other embodiments, the API has a particle size in range of about 1 micron to about 3 microns.

Aerosol solution formulation is less concerned with the particle size of the API. Bulk API may be used as long as the API forms a stable solution (i.e., no precipitate formation) in a suitable solvent. Although currently, the only solvent/cosolvent that has been approved by the Food and Drug Administration for use in oral inhalation formulation is ethanol, other potentially suitable solvents/cosolvents that may be used include propylene glycol, polyethylene glycol and water.

### Formulation for Oral Inhalation

The invention is directed to a pharmaceutical composition in unit dose form comprising rotigotine in an amount such that one or more unit doses are effective in the symptomatic treatment of one or more Parkinson's disease symptom(s) when administered to a patient. In some embodiments, the rotigotine is free base. In other embodiments, the rotigotine is a salt form. Suitable salt forms of rotigotine include, rotigotine glycolate, rotigotine lactate, rotigotine maleate, rotigotine palmitate, rotigotine pamoate, rotigotine propionate and rotigotine stearate. A preferred salt form of rotigotine for use in an oral inhalation formulation is rotigotine maleate.

Inhalation aerosols of drug formulation for delivery using a pressurized metered dose inhaler typically include excipients such as surfactants and other surface modifiers to increase the stability of the particles or to increase the deliverability of these drugs in an aerosol form. However, excipients such as surfactants and other surface modifiers have been associated with toxicity in the subject and other undesirable side effects. To avoid such toxicity problems, the drug formulation of the present invention is free of excipients such as surfactants and other surface modifiers.

The drug formulation may include one or more active pharmaceutical ingredient in any appropriate amount (singularly or in aggregate). In some embodiments, the API(s) may be selected to be in a certain concentration in order to achieve a desired concentration(s) after delivery into the subject or patient. In other embodiments, the API(s) may be selected to be in a certain concentration to conform to a certain dosing regimen or to achieve a certain desired effect.

Stability of a solution-based pMDI formulation can be determined by a variety of methods. One such method is to measure precipitate formation (if any) over time in different temperature/humidity conditions. Precipitate formation depends on the API interaction with the solvent and with the propellant for pMDI formulations. In some embodiments, a stable aerosol formulation will not have precipitate formation after 1 week at room temperature. In other embodiments, a stable aerosol formulation will not have precipitate formation after 1 week at 4-8° C.

Another desired characteristic of a solution-based pMDI formulation is the measure of the mass median aerodynamic diameter (MMAD) of the droplet size emitted from the device. The aerosol performance of a solution formulation is dependent on various factors such as propellant type (makeup), amount of solvent/cosolvent, API concentration and the container closure system. Unlike a suspension-based formulation, the API particle size is not a decisive factor for MMAD of a solution-based formulation since the API is solubilized in the propellant/solvent/cosolvent mixture. For proper deposition of the emitted formulation in the lung epithelium, a preferred range of MMAD is required. In some embodiments, the MMAD of the emitted formulation is between 1 micron and 5 microns. In other embodiments, the MMAD of the emitted formulation is between 2 microns and 3 microns.

### III. ORAL INHALATION DELIVERY SYSTEMS

The preferred embodiment of the rotigotine is delivered using inhalation therapy. Many preclinical and clinical studies with inhaled compounds have demonstrated that efficacy can be achieved both within the lungs and systemically. Moreover, there are many advantages associated with pulmonary delivery including rapid onset, the convenience of patient self-administration or with minimal assistance from a second person, the potential for reduced drug side-effects, ease of delivery by inhalation, the elimination of needles, and the like.

A wide variety of delivery methods/platforms are suitable for the practice of the invention Inhalation devices or other non-injectable devices are preferred devices and function by delivering an aerosol of the drug formulation into the subject or patient. These inhalation devices generally including a housing having a proximal end and a body portion. A mouthpiece or nose piece will typically be positioned at the proximal end.

### Nebulizers

Nebulizers generate an aerosol from a liquid, some by breakup of a liquid jet and some by ultrasonic vibration of the liquid with or without a nozzle. Liquid formulations are prepared and stored under aseptic or sterile conditions since they can harbor microorganisms. Liquid formulation can either be a suspension formulation or a solution formulation. The use of preservatives and unit dose packaging is contemplated. Additionally, solvents, detergents and other agents can be used to stabilize the drug formulation.

### Pressurized Metered Dose Inhalers

Pressurized metered dose inhalers or pMDIs, are an additional class of aerosol dispensing devices. pMDIs package the API formulation in a canister under pressure with a solvent and propellant mixture. Upon dispensed a jet of the mixture is ejected through a valve and nozzle and the propellant “flashes off” leaving an aerosol of the API formulation.

Propellants may take a variety of forms. In a non-limiting example, the propellant may be a compressed gas or a liquefied gas. Chlorofluorocarbons (CFCs) were once commonly used as liquid propellants, but have now been banned due to the negative impact on the earth's ozone layer. They have been replaced by the now widely accepted hydrofluorocarbon or hydrofluoroalkane (HFA) propellants. The most commonly used HFAs are 1,1,1,2-tetrafluoroethane, which is also referred to as 134a or HFA 134a; and 1,1,1,2,3,3,3-heptafluoropropane, which is also referred to as 227 or HFA 227, both available from Dupont, Solvay Chemicals, or Mexichem Fluor. In some cases, the propellant can be one HFA compound or a mixture of two or more HFA compounds.

In some embodiments, the propellant is selected from the group consisting of 1,1,1,2-tetrafluoroethane and 1,1,1,2,3,3,3-heptafluoropropane. In other embodiments, the propellant is 1,1,1,2-tetrafluoroethane. In some embodiments, the propellant is 1,1,1,2,3,3,3-heptafluoropropane. In some embodiments, the propellant is a mixture of 1,1,1,2-tetrafluoroethane and 1,1,1,2,3,3,3-heptafluoropropane.

The canister may contain multiple doses of the drug composition, although it is possible to have single dose canisters as well. The canister may include a valve, from which the contents of the canister may be discharged. In some embodiments, the valve is a metering valve. Aerosolized drug composition is dispensed from the pMDI by applying a force on the canister to push it into the receptacle, thereby opening the valve and causing the drug particles to be conveyed from the valve through the receptacle outlet. Upon discharge from the canister, the drug composition particles are atomized, forming an aerosol. pMDIs generally use propellants to pressurize the content of the canister and to propel the drug particles out of the receptacle outlet. In pMDIs, the drug composition is provided in liquid form, and resides within the canister along with the propellant.

In some instances, a manual discharge of aerosolized drug must be coordinated with inhalation, so that the drug composition particles are entrained within the inspiratory air flow and conveyed to the lungs. In other instances, a breath-actuated trigger, such as that included in the Tempo® inhaler (Allergan, Inc., Irvine, Calif.) may be employed that simultaneously discharges a dose of drug upon sensing inhalation. Such breath-actuated pMDI automatically discharges the drug composition aerosol at the appropriate time during inhalation by the user or subject. These devices are generally known as breath-actuated pressurized metered dose inhalers. Additionally, along with breath actuation, these devices may also be breath synchronized so as to discharge the bolus of the formulation at the height (largest volume) of the inspiratory breath. Breath-actuated pMDIs have additionally advantages including enhanced patient compliance and efficient, reliable dose-to-dose consistency that is independent of the inhalation flow rate. For example, the Tempo inhaler achieves these advantages by combining proprietary features such as a breath synchronized trigger and the flow control chamber and dose counter/lockout in a small, easy to use device. These advanced aerodynamic control elements are driven only by the patient's breath, avoiding expensive, power consuming electronics, resulting in an affordable, reliable, easy to use, and disposable platform. Because of the small size and option of breath-synchronization, a patient with Parkinson's disease is able to operate the device either to self-administer the formulation when needed, or with relatively little assistance from a second person who does not need to have formal medical training. In some variations, the pMDI can be fitted with a face piece or other adaptor to administer the drug for better and/or more efficient delivery.

All references cited herein are incorporated by reference in their entireties, whether previously specifically incorporated or not. The publications mentioned herein are cited for the purpose of describing and disclosing reagents, methodologies and concepts that may be used in connection with the present invention. Nothing herein is to be construed as an admission that these references are prior art in relation to the inventions described herein.

Although this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

The following examples serve to more fully describe and exemplify the above disclosed embodiments. It is understood that these examples in no way serve to limit the true scope of this disclosure, but rather are presented for illustrative purposes.

### IV. EXAMPLES

### Example 1

### In Situ Rotigotine Salt Screening

Rotigotine, bulk drug substance, was purchased from Chemagis (Perrigo API). A stock solution of rotigotine was prepared by dissolving rotigotine particles in ethanol at 10 mg/mL. With 1.5 mL rotigotine stock solution added to formulation bottles, each formulation bottle contained 15 mg rotigotine. For preparation of acid stock solutions, it was assumed that complete reaction or ion-pairing between rotigotine and acid. Based on the molecular weight and number of anions of each acid as listed in Table 1, acids stock solutions were prepared by dissolving the required amount in ethanol.

Each formulation bottle was filed with 1.5 mL of rotigotine stock solution and 1.0 ml, of acid stock solution. The formulation bottles (PET bottles) were then sealed with continuous valves and vortexed for 30 seconds to mix the solution well. Finally, 17.6 g of 1,1,1,2,3,3,3-heptafluoropropane (HFA227 (Mexichem)) to make a final volume of 15 mL of rotigotine formulation. All the bottles were hand shaken for 10 seconds.

All in situ salt screening samples were made as 1 mg/mL of rotigtine free base in 10% (w/w) ethanol in HFA 227. Acids corresponding to the salt forms in Table 1 were added according to the molar ratio with rotigotine for complete reaction. As 1 mg/mL was the minimal required dose at the time of experiment, all formulations were tested at this level to screen out non-solution salt formulations.

Among the acids tested for in situ salt screening, 5 formed white precipitate immediately upon preparation. These were the rotigotine citrate, fumarate, succinate, sulfate, tartrate, xinafoate salt formulations. These 5 rotigotine salt formulations were immediately ruled out as the precipitate formation was not desired. All clear solution samples were stored at room temperature (RT) for 1 week. After 1 week, 3 formed red precipitate. These were rotigotine acetate, benzoate and mesylate salt formulations. These 3 rotigotine salt formulations were also ruled out as precipitate formation is not desired. The rest of the samples remained as clear solutions (stable) at after another week at 5° C.

Out of the 7 acids that remained a clear solution after 1 week of storage, rotigotine maleate was selected as the lead candidate and suitable as a stable solution for oral pulmonary aerosol delivery. The selection criteria was based on the observation that the rotigotine maleate solution stayed in solution formulation at the minimum required concentration (1 mg/mL) and the maleate salt has been used in approved inhaled products, such as the Neohaler™

TABLE 1
In situ screening list of rotigotine salts.

| | | | | | Observation | |
|---|---|---|---|---|---|---|
| | | MW | | | 1 week at | 1 week at |
| Salt | Anion | (g/mol) | Structure | Immediate | RT | 5° C. |
| Acetate | 1 | 60 | 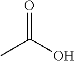 | solution | red precipitate | N/A |
| Benzoate | 1 | 122 | 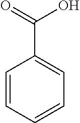 | solution | red precipitate | N/A |
| Citrate | 3 | 192 | 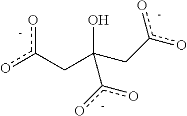 | white precipitate | N/A | N/A |
| Fumarate | 2 | 116 | 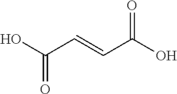 | white precipitate | N/A | N/A |
| Glycolate | 1 | 76 | 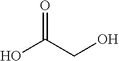 | solution | solution | solution |
| Lactate | 1 | 90 | 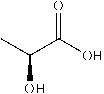 | solution | solution | solution |
| Maleate | 2 | 116 | 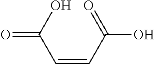 | solution | solution | solution |
| Mesylate | 1 | 96 | 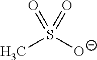 | solution | red precipitate | N/A |
| Palmitate | 1 | 284 | 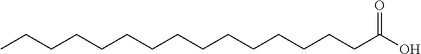 [SMILES: CCCCCCCCCCCCCCCC(=O)O] | solution | solution | solution |
| Pamoate | 2 | 388 | 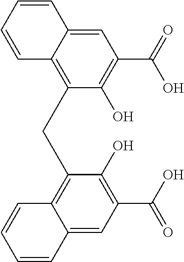 [SMILES: O=C(O)c1cc2ccccc2c(Cc2c(O)c(C(=O)O)cc3ccccc23)c1O] | solution | solution | solution |
| Propionate | 1 | 74 | 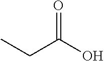 | solution | solution | solution |
| Stearate | 1 | 256 | 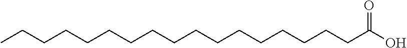 [SMILES: CCCCCCCCCCCCCCCCCC(=O)O] | solution | solution | solution |
| Succinate | 2 | 118 | 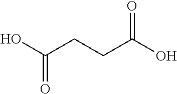 | white precipitate | N/A | N/A |
| Sulfate | 2 | 98 | 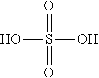 | white precipitate | N/A | N/A |
| Tartrate | 2 | 150 | 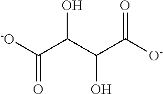 | white precipitate | N/A | N/A |
| Xinafoate | 1 | 188 | 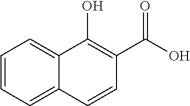 [SMILES: O=C(O)c1ccc2ccccc2c1O] | White precipitate | N/A | N/A |

We claim:

1. A pharmaceutical aerosol formulation comprising: (i) a dopamine agonist; (ii) a propellant; and (iii) a cosolvent, wherein the aerosol formulation is a stable solution formulation.

2. The pharmaceutical aerosol formulation of claim 1, wherein the formulation is stable at room temperature for at least a week.

3. The pharmaceutical aerosol formulation of claim 1, wherein the dopamine agonist is rotigotine or a pharmaceutically acceptable salt thereof.

4. The pharmaceutical aerosol formulation of claim 3, wherein the rotigotine is selected from the group consisting of rotigotine glycolate, rotigotine lactate, rotigotine maleate, rotigotine palmitate, rotigotine pamoate, rotigotine propionate, and rotigotine stearate.

5. The pharmaceutical aerosol formulation of claim 4, wherein the rogitotine is rotigotine maleate.

6. The pharmaceutical aerosol formulation of claim 1, wherein the propellant is selected from the group consisting of 1,1,1,2-tetrafluoroethane and 1,1,1,2,3,3,3-heptafluoropropane.

7. The pharmaceutical aerosol formulation of claim 6, wherein the propellant is 1,1,1,2,3,3,3-heptafluoropropane.

8. The pharmaceutical aerosol formulation of claim 6, wherein the propellant is a mixture of 1,1,1,2-tetrafluoroethane and 1,1,1,2,3,3,3-heptafluoropropane.

9. The pharmaceutical aerosol formulation of claim 1, wherein the cosolvent is selected from the group consisting of ethanol, propylene glycol, polyethylene glycol and water.

10. The pharmaceutical aerosol formulation of claim 9, wherein the cosolvent is ethanol.

11. The pharmaceutical aerosol formulation of claim 1, wherein the formulation is administered to a patient using a pressurized metered dose inhaler.

12. The pharmaceutical aerosol formulation of claim 11, wherein the pressurized metered dose inhaler is breath-actuated.

13. The pharmaceutical aerosol formulation of claim 1, wherein the formulation is administered to a patient using a nebulizer.

14. The pharmaceutical aerosol formulation of claim 1, wherein the concentration of the dopamine agonist is at least 1 mg/mL.

15. A pharmaceutical aerosol formulation comprising: (i) rotigotine or a pharmaceutically acceptable salt thereof; (ii) a propellant; and (iii) a cosolvent, wherein the aerosol formulation is a stable solution formulation and wherein the propellant is 1,1,1,2,3,3,3-heptafluoropropane and the cosolvent is ethanol.

16. The pharmaceutical aerosol formulation of claim 14, wherein the rotigotine is rotigotine maleate.
